(19) United States
(12) Patent Application Publication
(10) Pub. No.: US 2017/0119647 A1
(43) Pub. Date: May 4, 2017

(54) MINERAL-ENZYME COMPLEX FOR STRENGTHENING AND WHITENING TOOTH ENAMEL, ORAL HYGIENE COMPOSITION, AND TOOTHPASTE

(71) Applicants: Elena Yurievna BELOUS, Moscow (RU); Svetlana Alekseevna MALTABAR, Odintsovo (RU); Anna Zufarovna GALIMOVA, Aprelevka (RU)

(72) Inventors: Elena Yurievna BELOUS, Moscow (RU); Svetlana Alekseevna MALTABAR, Odintsovo (RU); Anna Zufarovna GALIMOVA, Aprelevka (RU)

(21) Appl. No.: 15/407,556

(22) Filed: Jan. 17, 2017

Related U.S. Application Data
(63) Continuation-in-part of application No. 14/422,985, filed on Feb. 20, 2015, now abandoned.

(30) Foreign Application Priority Data
Aug. 20, 2012 (RU) 2012135577

(51) Int. Cl. A61K 8/66; A61Q 11/00; C12N 9/18; A61K 8/24

(52) U.S. Cl. CPC A61K 8/66; A61K 8/24; A61Q 11/00; C12N 9/18; C12Y 301/0102; A61K 2800/92; A61K 2800/413

(57) ABSTRACT

The invention relates to the field of cosmetology, namely to mineral-enzyme complexes for tooth enamel strengthening and whitening, and also to compositions for oral hygiene comprising said complex, in particular, to toothpastes and other compositions. The described mineral-enzyme complex for tooth enamel strengthening and whitening is characterized by the fact that it contains calcium hydroxyapatite and tannase with the following component proportions: from 0.2 to 10 parts of tannase per 100 mass parts of calcium hydroxyapatite. The said complex may also additionally contain grapeseed extract. Besides, a variety of compositions for oral hygiene are disclosed that comprise the claimed mineral-enzyme complex. The invention makes it possible to improve consumer properties of the complex and compositions for oral cavity hygiene using the claimed mineral-enzyme complex: cleaning ability, whitening properties, reduction of gum inflammation and gum bleeding, improvement of desensitizing action while retaining remineralizing effect in respect of dental tissues.

### CROSS-REFERENCE TO RELATED APPLICATIONS

The present application is a Continuation-in-part of U.S. patent application Ser. No. 14/422,985 filed on Aug. 19, 2013, which is a National stage of PCT application PCT/RU2013/000716 filed on Aug. 19, 2013, which claims priority to Russian patent application RU 2012135577 filed Aug. 20, 2012, now Russian Federation patent No. 2494725, all of which Incorporated herein by reference.

### FIELD OF INVENTION

This invention relates to cosmetology, specifically, to a mineral-enzyme complex for strengthening and whitening tooth enamel, and also to compositions for oral cavity hygiene comprising such complex, in particular, toothpastes and other compositions.

### PRIOR ART

Patent literature comprises many publications disclosing the use of both individual minerals and mineral complexes in a variety of compositions for oral cavity hygiene, in particular, comprising calcium hydroxyapatite (calcium hydroxyphosphate).

Patent RU 2355380 claiming “A tooth elixir for the prevention and treatment of initial dental caries forms” discloses a mineral complex comprising calcium hydroxyapatite—a mineralizor contributing to the changing of tooth enamel mineralization kinetics, and sodium fluoride preventing the adsorption of organic substances and microorganisms on the surface of the enamel and participating in enamel prism formation.

A complex is also known comprising calcium hydroxyapatite and potassium citrate. Potassium citrate reduces the response of nerve fibres in exposed dental tubules to temperature, osmotic and tectile irritants. Calcium hydroxyapatite, in turn, fills dental tubules from the entrance aperture to the depth of the tubule, tightly sealing the entrance itself, which terminates the liquor outflow from dental tubules to the outside. As a result of that, intra-tubular osmotic pressure is restored, and pain syndrome in respect of external irritants is significantly reduced. The complex is designed to improve oral hygiene agent desensitizing action (see A. A. LEONTIEV et al, “Clinical Research of ‘Asepta Sensitive’ Anti-Sensitive Toothpaste”, Stomatology Today, No. 7 (87), 2009).

The same source also describes toothpaste, which, beside the described complex, also comprises thermal mud, marigold, melilot and calamus extracts, xylitol and papain enzyme, which taken in a complex, allow the paste to reader not just desensitizing, but also anti-inflammation and anti-plaque action.

The use of mineral complexes in oral cavity hygiene compositions allows to improve tooth mineralization, increase enamel resistance etc.

Disadvantages of known complexes include insufficient cleaning properties of the hygienic agents using such complexes, and inadequate tooth whitening.

These deficiencies may be eliminated by using individual enzymes or enzyme complexes in mouth cavity hygiene agents, disclosed quite broadly in the prior art.

Thus, for example, it is known that enzymes in the form of immobilized Bacillus subtilis proteinases, ensure cleaning and anti-inflammation action of mouth cavity hygienic agents (see patent RU2060030), while such enzyme as tannase, when used in oral cavity hygiene agents, helps remove color blemishes since it hydrolizes tannins contained in tea and coffee that cause tooth discoloration, (see specification to patent RU2416391).

Enzymes may be present as a set of substances. Patent RU2355420 discloses a complex of enzyme-active substances comprising papain, lidase, ribonuclease and lysozyme for the purpose of increasing cleaning, anti-microbial and anti-inflammation action, as well as restoration of the natural oral cavity microflora.

Also a toothpaste is disclosed herein using the above complex, comprising the following elements, mass %:

| | |
|---|---|
| silicon dioxide | 10.0-25.0 |
| glycerin | 5.0-10.0 |
| PEG 400 | 2.0-3.0 |
| xylitol | 5.0-6.0 |
| disodium EDTA | 0.2-1.0 |
| papain | 0.2-1.0 |
| lysozyme | 0.01-0.4 |
| lactolose | 2.0-6.0 |
| sodium carboxy-methylcellulose | 0.5-0.7 |
| carbomer | 0.2-0.4 |
| ribonuclease | 0.001-0.05 |
| lidase | 0.005-0.05 |
| sodium lauryl sarcosinate | 0.1-1.0 |
| sodium lauryl sulfate | 0.5-2.0 |
| cocamidopmpyl betaine | 0.5-3.0 |
| reduced glutathione | 0.001-0.01 |
| Extracts: camomile, parsley | 0.05-1.0 |
| grapeseed | 0.05-1.0 |
| glucospheres | 0.5-3.0 |
| tetrapotassium pyrophosphate | 2.0-5.0 |
| tetrasodium pyrophosphate | 0.5-2.0 |
| disodium pyrophosphate | 0.3-1.0 |
| sodium saccharinate | 0.1-0.6 |
| citric acid | 0.1-0.4 |
| sodium citrate | 0.1-2.0 |
| fir tree extract | 0.05-1.0 |
| sage extract | 0.05-1.0 |
| chondroitin sulphate | 0.1-0.5 |
| betaine | 2.0-4.0 |
| aminefluoride | 0.19-1.95 |
| sodium fluoride | 0.1-0.3 |
| sodium methyl paraben | 0.1-0.5 |
| sodium propyl paraben | 0.1-0.3 |
| titanium dioxide | 0.3-0.5 |
| flavoring agent | 0.05-2.0 |
| polyvinyl pyrrolidone | 0.1-0.5 |
| demineralized water | 10.84-66.893 |

This complex and toothpaste using the same have whitening and antimicrobial properties, but do not exert a mineralizing effect on the hard tissues of the teeth, strengthen the enamel but weakly, and do not possess sufficient anti-inflammatory action.

As follows from the above sources, calcium hydroxyapatite based complexes provide a solution to the problem of improving dental tissue mineralization, while enzyme based complexes improve teeth whitening, i.e. they are rather narrowly specialized and do not possess the entire range of protective properties. In particular, none of the complexes under study or any dental hygiene agents containing the same, possesses a combined anti-inflammatory and blood-stopping action along with teeth cleaning and whitening.

### DISCLOSURE OF THE INVENTION

The objective of this invention is to provide a mineral-enzymatic complex for hygienic protection of tooth enamel and oral cavity, which having both:
1) cleaning and
2) whitening ability, the use of which
3) reduces inflammation and bleeding of gums while
4) improving sensitivity, desensitizing effectiveness with remineralizing effect preserved.

The set objective is achieved through the use of a mineral-enzyme complex for tooth enamel strengthening and whitening, comprising calcium hydroxyapatite and tannase with the following component proportions: 0.2 to 10 parts of tannase per 100 mass parts of calcium hydroxyapatite.

In particular embodiments of the invention, the set objective is achieved by adding grapeseed extract with the following component proportions; 0.2 to 10 parts of tannase and up to 100 mass parts of grapeseed extract per 100 mass parts of calcium hydroxyapatite.

The set objective is also achieved by means of a composition for oral cavity hygiene comprising the above-said mineral-enzyme complex for tooth enamel strengthening in an effective quantity and an appropriate medium.

In particular embodiments of the invention, the said composition may represent a toothpaste, rinsing composition, foaming rinsing composition or a chewing gum.
1) The set objective is also achieved by means of a toothpaste for oral cavity hygiene comprising the said mineral-enzyme complex for tooth enamel strengthening in an effective quantity and an appropriate medium, said toothpaste additionally comprising substances selected from a group containing abrasive substances, moisturizers, thickeners, surfactants and solvents.

The toothpaste may additionally comprise substances selected from a group containing colorants, preservatives, flavoring agents, anti-oxidizing agents, mineralizing agents, vitamins and mixtures thereof.

In particular embodiments of the invention, a toothpaste comprises a medium containing water as a solvent, silicium dioxide, as an abrasive substance, sodium carboxymethylcellulose as a thickener, at least one moisturizer selected from a group comprising glycerin, sorbitol and polyethylene glycol, and sodium lauryl sarcosinate as a surfactant, with the following component proportions, mass %:

| | | |
|---|---|---|
| 1. | Mineral-enzyme complex | 1.50-30.00 |
| 2. | Silicium dioxide | 1.00-60.00 |
| 3. | Sodium carboxy-methylcellulose | 0.50-10.00 |
| 4. | Moisturizer | 0.50-7000 |
| 5. | Sodium lauryl sarcosinate | 0.01-10.00 |
| 6. | Water | the rest. |

2) That toothpaste may additionally contain xanthan gum as a thickener in a quantity not exceeding 3 mass %.

3) The toothpaste may additionally contain substances selected from a group containing foam stabilizers, colorants, preservatives, flavoring agents, anti-oxidizing agents, mineralizing agents, anti-inflammatory binding agents, aseptics, anti-caries agents and mixtures thereof with the following proportions, mass %:

| | | |
|---|---|---|
| 1. | Foam stabilizers | max. 5.00 |
| 2. | Colorants | max. 5.00 |
| 3. | Preservatives | max. 0.30 |
| 4. | Flavoring agents | max. 3.00 |
| 5. | Anti-oxidizing agents | max. 5.00 |
| 6. | Mineralizing agents | max. 10.00 |
| 7. | Anti-inflammatory binding agents | max. 10.00 |
| 8. | Aseptics | max. 1.00 |
| 9. | Anti-caries agents | max. |

The essence of the invention is as follows.

Calcium hydroxyapatiter possesses high adhesion and resorption properties in respect of all types of tissue, both bone and epithelial. As a result of resorption, soft tissues develop a protective coating, and the process of regeneration/granulation is promoted with the generation of new cells and tissues at the place of injury or inflammation.

Tannase is an esterase group enzyme. This enzyme catalyzes hydrolitic cleavage of tannine. There is information in literature that tannase is strictly specific in its action: it disintegrates complex ethers whose acidic component contains at least two phenolic hydroxyls.
1) We assume that tannase is capable of catalyzing processes in the mouth cavity with the production of anti-bacterial substances that enhance, in a non-specific manner, anti-inflammatory properties of calcium hydroxyapatite, at the same time ensuring intensive teeth whitening.

For the claimed complex, it is advisable that amorphous calcium hydroxyapatite with a particle size of 20-80 nm be used. Such calcium, hydroxyapatite form does not have abrasive properties while possessing improved biocompatibility with dental tissue.

Research has shown that all the above mineral-enzyme complex, components should be gathered together is certain proportions in the complex; in that case, there is a synergetic effect which provides an opportunity to improve tooth enamel and periodontal tissue conditions in a non-liner fashion.

Beyond the claimed proportions of the components, the claimed technical result is not achieved.

The mineral-enzyme complex may contain op to 100 mass parts of grapeseed extract, which further enhances anti-inflammatory properties of the oral cavity hygiene composition.

An oral cavity hygiene composition, in the broadest sense, shall mean composition comprising the above-said mineral-enzyme complex for tooth enamel strengthening as an active ingredient in an effective quantity and an appropriate medium.

An effective mineral-enzyme complex quantity may be different depending on the type of composition, particular sets of ingredients or a combination of substances in the complex etc.

Such effective quantity in any individual case may be determined by a professional versed in the field by means of ordinary experiments.

For certain embodiments of the invention (toothpaste) such quantity has been identified by us and cited in the claims of the invention.

An appropriate medium shall mean such that enables to obtain a desired oral cavity hygiene composition in the form of e.g. toothpaste, rinse liquid, tooth, powder, gel, foaming rinse, chewing gum etc. In that sense, a composition under the present invention may be represented by a liquid, i.e. a solution of ingredients, e.g. a mouth rinsing liquid; or it may be semi-hard, as for example toothpaste or tooth cleaning gel; or it may be hard, e.g. chewing gum.

If the composition hereunder is a liquid, e.g. a month rinsing, liquid, then an appropriate medium is represented, as a rule, by a water-glycerin mixture (traditional rinse) or glycerin (high sensitivity denial rinse or applications). Solubilizers and other useful additives may be added to the rinse composition. In particular, the base of the foaming rinse, beside solubilizers, additionally contains surfactants and a foam stabilizing agent.

If the composition is a chewing gum, then an appropriate medium comprises synthetic or natural polymers with plastifiers, flavoring agents, preservatives, sweeteners and other additives.

If the composition is a toothpaste, then an appropriate medium shall comprise solvents, thickeners, surfactants, abrasive substances, emulsifiers, solubilizers, moisturizers, sweeteners, flavoring agents, preservatives and mixtures thereof.

An analysis of prior art shows that for a toothpaste, the abrasive content fluctuates from about 5 to about 60 mass %, which corresponds to the abrasive content of the claimed toothpaste. Abrasive substances preferable for use in the present invention include silicium dioxide based materials, represented in the invention examples below by Sorbosil (by PQ Corporation) and Tixosil (by Rhodia). However, that list of abrasives is not exhaustive, it being possible to use such substances as abrasive agents as aluminum oxide, calcium carbonate, sodium metaphosphate, potassium metaphosphate, tricaicium phosphate, dehydrated dicalcium phosphate, aluminum silicate, calcined aluminum oxide, bentonite or other silicium based materials or combinations thereof.

An appropriate medium applicable for the obtaining of the composition hereunder in the form of a paste or foaming rinse may comprise a moisturizer. A moisturizer is preferably represented by sorbitol, glycerin and/or polyethylene glycol PEG 400; however, other moisturizers and mixtures thereof with a molecular mass in the range of 200-1000 may also be used. In prior art technical solutions, moisturizer concentrations are usually in the range of from about 0.5 to about 70% of the composition mass.

As a rule, thickeners are present in oral hygiene compositions in quantitities of up to 10 mass %. Thickeners include natural and synthetic resins and colloids. In the present invention, the thickener is represented by sodium carboxy-methylcellulose; besides, the thickener functions are also performed by the above discussed silicium dioxide.

Any of the above listed compositions may additionally contain any appropriate flavoring or sweetening substances.

Toothpastes may additionally comprise a variety of other substances including preservatives, such as methylparaben, sodium methylparaben and chlorophyllic compounds. Such auxiliary substances shall be introduced into compositions hereunder in quantities that do not have an adverse affect on the desired properties and characteristics.

The introduction of emulsion stabilizers in the paste ensures a dispersed state of fats and oils in water emulsions.

Requirements to stabilizers are as follows: ensuring composition stability, inertness to other composition components, no irritating action, non-toxicity, no offensive odor. All of the above requirements are satisfied by Polydon-A, tetrasodium glutamate diacetate and disodium EDTA (Trilon BD), used in particular embodiments of the invention.

A toothpaste may comprise surfactants and, in particular, anionic surfactants, such as sodium lauryl sulphate, sodium myristoyl sarcosinate, cocamidopropyl betaine and sodium lauryl sarcosinate, which possess a multi-functional action:; solubilizing, dispensing and moisturizing. Moreover, their function is to form emulsions with other components of oral cavity hygiene compositions, including those with flavoring additives. Beside the above-listed surfactants, other anionic surfactants may also be used, as well as cationic, non-ionic, amphoteric and zwitter-ionic surfactants, or mixtures thereof.

The effect of a surfactant consists in its contribution to the foaming of a toothpaste. The introduction of surfactant with a high degree of foaming contributes to the sensation of effective oral cavity cleaning.

Compositions for oral cavity hygiene according to the present invention may be obtained by mixing the ingredients.

### EXAMPLES

The examples provided describe and demonstrate options falling within the scope of this invention. These examples are given for illustration purposes only, and should not be construed as limiting this invention.

### Example I

### A General Method of Preparing a Composition for Dental Care and Oral Hygiene Based on the Mineral-Enzymatic Complex

### 1. Preparation Actions:

2) Prepare a dispersion of a thickening agent in a solvent
3) If necessary, prepare a premix containing a flavor and menthol, essential oils, oil-soluble active components.
4) If necessary, prepare a water-based surfactant solution.
5) If necessary, prepare a premix of active components.

### 1. Cooking Process:

2.1 Load successively into the reactor:
water,
moisturizers,
thickener dispersion in a solvent prepared according to par. 1.1.

2.2 Mix, stir and homogenize in vacuum until homogeneous state for 15-20 minutes.

2.3 Load successively into the reactor:
active additives, including a premix of active components made according to par. 1.4,
dyes.

2.4 Mix, stir and homogenize in vacuum until homogeneous state for 15-20 minutes.

2.5 Take a sample to determine viscosity (a sample should be homogeneous, without lumps and crumbs).

2.6 Load an abrasive component in small portions into the reactor and, alter wetting the abrasive component, homogenize the mass until homogeneous under vacuum.

2.7 Load successively into the reactor:
Active components sensitive to temperatures above 38° C.,
a premix made according to par. 1.2.

2.8 Mix, stir aid homogenize in vacuum until homogeneous state for 5-10 minutes.

2.9 Load into the reactor a water-based surfactant solution made according to par. 1.3.

2.10 Mix, stir and homogenize in vacuum until homogeneous state for 15-25 minutes.

3. Take a sample to determine physico-chemical and organoleptic indicators for conducting a microbiological analysis.

### Example II

### A Method of Preparing Compositions for Dental Care and Oral Hygiene Based on a Mineral-Enzymatic Complex

### II.1 Process #1

### 1. Preparation Actions:

2) Disperse sodium carboxymethyl cellulose and Stevia in PEG-400.
3) Dissolve Trilon BD in water,
4) Dissolve citric acid in water.
5) Mix polidon A with, water.
6) Dissolve Thymol and Biosol in a flavoring agent and, if necessary, heat to 40° C.
7) Dissolve tannase in water.
8) Prepare a suspension of 15% amorphous hydroxyapatite with 20-80 nm particle size (15% HAP),

### 1. Cooking Process:

2.1 Load successively into the reactor:
sorbitol
glycerine,
suspension according to par. 1.7.

2.2 Mix, stir and homogenize.

2.3 Load successively into the reactor:
dispersion according to par. 1.1,
water.

2.4 Mix, stir and homogenize.

2.5 Load successively into the reactor;
solution according to par. 1.2,
sodium monofluorophosphate,
xylitol,
methylparaben sodium salt,
solution according to par. 1.3.

2.6 Mix, stir and homogenize.

2.7 Load successively into the reactor:
solution according to par. 1.4,
olaflur.

2.8 Mix, stir and homogenize,

2.9 Take a sample to determine viscosity (a sample should be homogeneous, without lumps and crumbs.

2.10 Load successively into the reactor:
silicone dioxide (Sorbosil TC15),
titanium dioxide,
Sorbosil AC43.

2.11 Mix, stir and homogenize.

2.12 Load successively in to the reactor:
Sorbosil AC39.

2.13 Mix, stir and homogenize.

2.14 Load successively into the reactor:
anethole,
eucalyptol,
solution according to par. 1.5,
Vitamin E,
CO2 extract of grape-seed,
solution according to par. 1.6.

2.15 Mix, stir and homogenize.

2.16 Take a sample to determine viscosity (a sample should be homogeneous, without lumps and crumbs).

2.17 Load successively into the reactor:
33% solution of cocamidopropyl betaine
30% solution of sodium lauroyl sarcosinate.

2.18 Mix, stir and homogenize.

3. Take a sample to determine pH and viscosity values.

Color: white.

pH 25%: 6.0-7.0.

### II.2 Process #2

### 1. Preparation Actions:

2) Disperse sodium carboxymethyl cellulose and Stevia in PEG-400.
3) Dissolve Triton BD in water.
4) Dissolve citric acid in water.
5) Dissolve dry extracts of bergenia and scullcap (Scutellaria) in water at to 40-45° C.
6) Dissolve Thymol and Biosol in a flavoring agent and, if necessary, heat to 40° C.
7) Dissolve tannase and extract of Azadirachta indica in water.
8) Prepare a suspension of 15% amorphous hydroxyapatite with 20-80 nm particle size (15% HAP).

### 1. Cooking Process:

2.1 Load successively into the reactor:
sorbitol,
glycerine,
suspension according to par. 1.7.

2.2 Mix, stir and homogenize.

2.3 Load successively into the reactor:
dispersion according to par. 1.1,
water.

2.4 Mix, stir and homogenize.

2.5 Load successively into the reactor:
solution according to par. 1.2,
xylitol,
methylparaben sodium salt,
solution according to par. 1.3.

2.6 Mix, stir and homogenize.

2.7 Load successively into the reactor:
solution according to par. 1.4,

2.8 Mix, stir and homogenize.

2.9 Take a sample to determine viscosity (a sample should be homogeneous, without lumps and crumbs).

2.10 Load successively into the reactor:
silicone dioxide (Sorbosil TC15),
titanium dioxide,
Sorbosil AC43,

2.11 Mix, stir and homogenize.

2.12 Load successively into the reactor:
Sorbosil AC39.

2.13 Mix, stir and homogenize.

2.14 Load successively into the reactor:
anethole,
eucalyptol,
solution according to par. 1.5,
Vitamin E,
CO2 extract of grape-seed,
solution according to par. 1.6.

2.15 Mix, stir and homogenize.

2.16 Take a sample to determine viscosity (the sample should be homogeneous, without lumps and crumbs).

2.17 Load successively into the reactor:
33% solution, of cocamidopropyl betaine,
30% solution of sodium lauroyl sarcosinate.

2.18 Mix, stir and homogenize.

3. Take a sample to determine pH and viscosity values.

Color: light-beige.

pH 25%: 6.0-7.0.

### II.3 Process #3

### 1. Preparation Actions:

2) Disperse sodium carboxymethyl cellulose and Stevia in PEG-400.
3) Dissolve Triton BD in water.
4) Dissolve citric acid in water.
5) Dissolve Thymol and Biosol in a flavoring agent and, if necessary, heat to 40° C.
6) Dissolve tannase in water.
7) Prepare a suspension of 15% amorphous hydroxyapatite with 20-80 nm particle size (15% HAP).

### 1. Cooking Process:

2.1 Load successively into the reactor:
sorbitol,
glycerine,
suspension according to par. 1.6.

2.2 Mix, stir and homogenize.

2.3 Load successively into the reactor:
dispersion according to par. 1.1,
water.

2.4 Mix, stir and homogenize.

2.5 Load successively into the reactor:
solution according to par; 1.2,
sodium monofluorophosphate,
xylitol,
methylparaben sodium salt,
solution according to par. 1.3.

2.6 Mix, stir and homogenize.

2.7 Load successively in to the reactor:
calcium lactate,
olaflur.

2.8 Mix. stir and homogenize.

2.9 Take a sample to determine viscosity (the sample should be homogeneous, without lumps and crumbs).

2.10 Load successively into the reactor:
silicone dioxide (Sorbosil TC15),
titanium dioxide,
Sorbosil AC43,

2.11 Mix, stir and homogenize.

2.12 Load successively into the reactor:
Sorbosil AC39.

2.13 Mix, stir and homogenize.

2.14 Load successively into the reactor:
anethole,
eucalyptol,
solution according to par. 1.4,
Vitamin E,
CO2 extract of grape-seed,
solution according to par. 1.5.

2.15 Mix, stir aid homogenize.

2.16 Take a sample to determine viscosity (the sample should be homogeneous, without lumps and crumbs).

2.17 Load successively into the reactor:
33% solution of cocamidopropyl betaine,
30% solution of sodium lauroyl sarcosinate.

2.18 Mix, stir and homogenize.

3. Take a sample to determine pH and viscosity values.

Color: white

pH 25% 6.0-7.0.

### Example III

### Toothpaste Compositions

The toothpaste compositions, the formulations of which are disclosed in Table 1, contain a mineral-enzymatic complex based on tannase and amorphous calcium hydroxyapatite with 20-80 nm particle size, the said complex strengthening and whitening tooth enamel reducing tooth sensitivity; having anti-inflammatory and hemostatic properties.

TABLE 1
Toothpaste compositions.

| | | Toothpaste compositions, mass % | | | | | |
|---|---|---|---|---|---|---|---|
| | | Composition | Composition | Composition | Composition | Content | |
| Ser. # | Component | A¹ | B² | C³ | D⁴ | range | Function |
| 1. | Mineral- | 6.06 | 1.52 | 10.05 | 11.10 | 1.50-30.00 | Source of mineral substances |
| | enzyme | | | | | | and tannase enzyme; |
| | complex | | | | | | contributes to strengthening |
| | | | | | | | and whitening of enamel, |
| | | | | | | | reduction of tooth sensitivity, |
| | | | | | | | possesses anti-inflammatory |
| | | | | | | | and blood-stopping |
| | | | | | | | properties |
| 2. | Sorbitol | 30.00 | 20.00 | 20.00 | 20.0 | 1.00-60.00 | Moisturizer. |
| | 70% | | | | | | Influences the product |
| | | | | | | | texture imparting special |
| | | | | | | | softness and plasticity |
| | | | | | | | thereto. |
| 3. | Glycerin | 10.00 | 5.00 | 5.00 | 5.00 | 0.50-70.00 | Moisturizer. |
| | (99.7%) | | | | | | Contributes to obtaining a |
| | | | | | | | plastic, thixotropic mass, |
| | | | | | | | stabilizes foam, improves |
| | | | | | | | toothpaste flavor. |
| 4 | Polyethylene | — | 5.00 | 5.00 | — | 0.10-20.00 | Moisturizer. |
| | glycol | | | | | | Influences the product |
| | PEG 400 | | | | | | texture imparting special |
| | | | | | | | softness and plasticity |
| | | | | | | | thereto. |
| 5. | Silicium | 10.0 | 16.0 | 10.0 | 15 | 1.00-60.00 | Thickener and/or abrasive |
| | dioxide | | | | | | |
| 6. | Sodium | 1.00 | 1.5 | 1.50 | 1.50 | 0.5-10.00 | Thickener, structure-forming |
| | carboxy- | | | | | | agent |
| | methylcellulose | | | | | | |
| 7. | Xanthan | 0.10 | — | — | — | 0.02-3 | Thickener, structure-forming |
| | gum | | | | | | agent |
| 8. | Sodium | 4.00 | — | — | — | 2.00-5.00 | Surfactant |
| | myristoyl | | | | | | |
| | sarcosinate | | | | | | |
| 9 | Cocamido | — | 1.00 | — | — | 0.01-10.00 | Surfactant |
| | propyl | | | | | | |
| | betaine | | | | | | |
| 10. | Sodium | — | 1.00 | 3.0 | — | 0.01-10.00 | Surfactant |
| | lauryl | | | | | | |
| | sarcosinate | | | | | | |
| 11. | Sodium | — | — | — | 2.0 | 0.01-10.00 | Surfactant |
| | lauryl | | | | | | |
| | sulphate | | | | | | |
| | (sodium | | | | | | |
| | coco- | | | | | | |
| | sulphate) | | | | | | |
| 12. | Stevia | 0.20 | — | 0.20 | 0.20 | 0.05-3 | Sweetener |
| | extract | | | | | | |
| 13. | Xylitol | — | 0.50 | 1.0 | — | 0.01-1 | Sweetener |
| 14. | Sucralose | | 0.05 | | | 0.01-1 | Sweetener |
| 15. | Flavoring | 0.50 | 1.0 | 1.0 | 1.0 | 0.05-3 | Flavoring additive |
| | agent | | | | | | |
| 16. | Sodium | 0.30 | 0.25 | | 0.300 | 0.05-0.3 | Preservative |
| | methylparaben | | | | | | |
| 17. | Titanium | | 0.10 | 0.10 | 0.10 | 0.01-5.00 | Whitening agent, colorant |
| | dioxide | | | | | | |
| 18. | Anise | 0.150 | — | — | — | 0.01-1 | Polyphenol, aseptic |
| | camphor | | | | | | |
| 19. | Eucalyptol | 0.10 | — | — | — | 0.01-1 | Polyphenol, aseptic |
| 20. | Thymol | 0.08 | — | — | — | 0.01-1 | Polyphenol, aseptic |
| 21. | Isopropyl | 0.100 | — | 0.10 | — | 0.01-0.2 | Aseptic, caries prevention |
| | methylphenol | | | | | | |
| 22. | Sodium | — | — | 0.50 | — | 0.01-10.00 | Anti-caries agent |
| | monofluorophosphate | | | | | | |
| 23. | Aminefluoride | — | — | 1.00 | — | 0.01-10.00 | Anti-caries agent |
| 24. | Vitamin E | 0.10 | — | — | 0.10 | 0.01-5.00 | Anti-oxidizing agent, anti- |
| | | | | | | | inflammatory action |
| 25. | Tetrasodium | 0.50 | — | — | | 0.01-5.00 | Foam stabilizer, chelating |
| | glutamate | | | | | | and whitening agent |
| | diacetate | | | | | | |
| 26. | Polydon A | — | — | 0.90 | — | 0.01-5.00 | Foam stabilizer, whitening |
| | | | | | | | agent |
| 27. | Trilon BD | — | 0.05 | 0.05 | — | 0.01-5.00 | Foam stabilizer, chelating |
| | (disodium | | | | | | and whitening agent |
| | EDTA) | | | | | | |
| 28. | Calcium | — | — | — | 1.00 | 0.01-10.00 | Mineralizing agent |
| | lactate | | | | | | |
| 29. | Citric acid | 0.10 | 0.20 | — | — | 0.0001-0.50 | Acidity regulating agent |
| 30. | Dry | — | 0.10 | — | — | 0.01-10.00 | Anti-inflammatory, binding, |
| | skullcap | | | | | | blood-stopping agent |
| | extract | | | | | | |
| 31. | Dry | — | 0.10 | — | — | 0.01-10.00 | Anti-inflammatory, binding, |
| | bergenia | | | | | | blood-stopping agent |
| | extract | | | | | | |
| 32. | Neem | — | 0.05 | — | — | 0.005-5.00 | Antibacterial agent |
| | extract | | | | | | |
| 33. | Water | Up to 100% | | | | | Solvent |

¹In the mineral-enzymatic complex containing 0.2 weight parts of tannase per 100 weight parts of hydroxyapatite.
²In the mineral-enzymatic complex containing 2 weight parts of tannase per 100 weight parts of hydroxyapatite.
³In the mineral-enzymatic complex containing 0.5 weight parts of tannase per 100 weight parts of hydroxyapatite.
⁴In the mineral-enzymatic complex containing 2 weight parts of tannase per 100 weight parts of hydroxyapatite.

### Example IV

### Mouthwash Composition

TABLE 2
Mouthwash composition.

| Ser. | | Composition, | |
|---|---|---|---|
| # | Composition component | mass % | Function |
| 1 | Mineral-enzyme complex⁵ | 1.60 | Contributes to strengthening and |
| | | | whitening of enamel, reduction of |
| | | | tooth sensitivity, possesses anti- |
| | | | inflammatory and blood-stopping |
| | | | properties |
| 2 | Glycerin (99.7%) | 5.00 | Moisturizer, solvent |
| 3 | Triton BD (disodium | 0.05 | Stabilizer, chelating and |
| | EDTA) | | whitening agent |
| 4 | Sucralose | 0.05 | Sweetener |
| 5 | Sodium methylparaben | 0.30 | Preservative |
| 7 | Xanthan gum | 0.50 | Thickener, structure-forming agent |
| 8 | Calcium lactate | 1.00 | Mineralizing agent |
| 9 | Polyethylene glycol PEG 400 | 5.00 | Moisturizer. |
| 10 | Thymol 001-017 | 0.08 | Polyphenol, aseptic |
| 11 | PEG-40 hydrogenized | 1.00 | Moisturizer, solubilizer |
| | castor oil | | |
| 13 | Vitamin E | 0.05 | Anti-oxidizing agent, anti- |
| | | | inflammatory action |
| 14 | Flavoring agent | 0.30 | Flavoring additive |
| 15 | Anise camphor | 0.10 | Polyphenol, aseptic |
| 16 | Eucalyptol | 0.05 | Polyphenol, aseptic |
| 18 | Citric acid (dry) | 0.20 | Acidity regulating agent |
| 19 | Purified water | Up to 100% | Solvent |

⁵In the mineral-enzymatic complex containing 10.0 weight parts of tannase per 100 weight parts of hydroxyapatite.

### Example V

### Composition for Rinsing and Application for Sensitive Teeth

TABLE 3
Composition for rinsing and application for sensitive teeth.

| Ser. | Composition | Composition, | |
|---|---|---|---|
| # | component | mass % | Function |
| 1 | Mineral- | 20.15 | Source of mineral substances and |
| | enzyme | | tannase enzyme; contributes |
| | complex⁶ | | to strengthening and |
| | | | whitening of enamel, |
| | | | reduction of tooth sensitivity, |
| | | | possesses anti-inflammatory |
| | | | and blood-stopping properties |
| 2 | Glycerin | Up to 100% | Moisturizer, |
| | (99.7%) | | Contributes to obtaining a plastic, |
| | | | thixotropic mass, stabilizes foam, |
| | | | improves toothpaste flavor. |
| 3 | Japanese | 1.00 | Antibacterial agent |
| | honeysuckle | | |
| | extract | | |
| 4 | p-methoxy- | 0.10 | Antibacterial agent |
| | benzoic | | |
| | acid | | |
| 5 | Glyceryl | 0.10 | Antibacterial agent |
| | monocaprilate | | |
| | (Cosphaderm | | |
| | GMCY) | | |

⁶In the mineral-enzymatic complex containing 0.25 weight parts of tannase per 100 weight parts of hydroxyapatite.

### Example VI

### Foam Mouthwash Composition

TABLE 4
Foam mouthwash composition.

| | | Compo- | |
|---|---|---|---|
| | Composition | sition, | |
| Ser. | component | mass % | Function |
| | Mineral-enzyme | 0.71 | Source of mineral substances |
| | complex⁷ | | and tannase enzyme; |
| | | | contributes to strengthening and |
| | | | whitening of enamel, |
| | | | reduction of tooth sensitivity, |
| | | | possesses anti-inflammatory, |
| | | | and blood-stopping properties |
| | Sorbitol 70% | 10.00 | Moisturizer. |
| | | | Influences the product |
| | | | texture imparting |
| | | | special softness |
| | | | and plasticity thereto. |
| | Triton BD | 0.10 | Stabilizer, chelation |
| | (disodium EDTA) | | and whitening agent |
| | Sodium | 0.25 | Preservative |
| | methylparaben | | |
| | L-arginine | 0.100 | Acidity regulating agent |
| | Sucralose | 0.05 | Sweetener |
| | Liquorice extract | 0.05 | Anti-inflammatory and |
| | | | anti-caries agent |
| | Sodium lauryl | 4.00 | Surfactant |
| | sarcosinate | | |
| | Polyvinyl | 0.10 | Whitening agent, foam stabilizer |
| | pyrrolidone/ | | |
| | vinylacetate | | |
| | Flavoring agent | 1.00 | Flavoring additive |
| | PEG-40 | 1.00 | Moisturizer, solubilizer |
| | polysorbate-20 | 1.00 | Moisturizer, solubilizer |
| | carragheenan | 0.05 | Foam stabilizer |
| | Citric acid | 0.20 | Acidity regulating agent |
| | Enzyme complex: | 0.50 | Antibacterial agent |
| | lactoferrin, | | |
| | lactoperoxidase, | | |
| | glucose oxidase, | | |
| | potassium | | |
| | thiocyanate, | | |
| | glucose | | |
| | pentaacetate | | |
| | Purified | Up to | Solvent |
| | water | 100% | |

⁷In the mineral-enzymatic complex containing 0.25 weight parts of tannase per 100 weight parts of hydroxyapatite.

### Example VII

### Study of Effectiveness of the Toothpastes According to the Invention

The toothpastes prepared (compositions A, B, C, and D from Table 1) and the reference standard D′ without tannase were tested as follows.

Check-up dental examinations were to identify the condition of oral cavity hygiene, hard tooth tissue, periodontal tissue and mouth mucosa by the following criteria.

### Material and Methods of the Study.

In approval of the claimed toothpaste subjects with sensitive teeth participated. The total of 480 sensitive teeth were found in the study subjects, in average 6 sensitive teeth per subject. Depending on the toothpaste variant applied, the study subjects were divided into groups:

Group 1—the group participants used a paste with composition A;

Group 2—the group participants used a paste with composition B:

Group 3—the group participants used a paste with composition C;

Group 4—the group participants used a paste with composition D;

Group 5—the group participants used a paste with composition D′.

It was a double-blind randomized study.

The toothpastes were used by the study subjects themselves twice daily (in the morning and at night) for one month. Following the initial examination, repeated examinations were performed on a weekly basis for 4 weeks.

### 1) The Study of Cleaning Action and Effect.

To determine the cleaning action, the study authors used Green-Vermillion hygienic factor and reagent tablets by PARO.

According to Green-Vermillion hygienic factor, the cleaning effect was determined by the following formula:

Effect (%)=[100×(OHI₀−OHIₙ)]/OHI₀

where OHI₀ is index value at research start, prior to hygienic procedure;

OHIₙ is index value after n weeks of research, at the last check-up prior to hygienic procedure.

Table 5.1 summarizes data for 4-weeks' dynamics of Green-Vermillion hygienic factor, while Table 5.2 provides data as changes in cleaning effectiveness according to Green-Vermillion hygienic factor.

TABLE 5.1
Dynamics of Green-Vermillion hygienic factor.

| | Distribution by | Examination period | | | | |
|---|---|---|---|---|---|---|
| Ser. # | group | Start | Week 1 | Week 2 | Week 3 | Week 4 |
| 1 | Composition A | 3.70 | 3.21 | 2.81 | 2.43 | 2.22 |
| 2 | Composition B | 3.72 | 3.23 | 2.84 | 2.43 | 2.27 |
| 3 | Composition C | 3.80 | 3.32 | 2.93 | 2.57 | 2.21 |
| 4 | Composition D | 3.69 | 3.28 | 2.89 | 2.49 | 2.26 |
| 5 | Composition D′ | 3.78 | 3.67 | 3.52 | 3.45 | 3.33 |

TABLE 5.2
Change in the cleaning effect
according to the Green-Vermillion factor

| | Distribution | Examination period | | | |
|---|---|---|---|---|---|
| Ser. # | by group | Week 1 | Week 2 | Week 3 | Week 4 |
| 1 | Composition A | 13.24% | 24.05% | 34.32% | 40.00% |
| 2 | Composition B | 13.17% | 23.66% | 34.68% | 38.98% |
| 3 | Composition C | 12.63% | 22.89% | 32.37% | 41.84% |
| 4 | Composition D | 11.11% | 21.68% | 32.52% | 38.75% |
| 5 | Composition D′ | 2.91% | 6.88% | 8.73% | 11.90% |

### 1) Study of Sensitivity (Tactile and Heat Samples).

Intensity of hyperesthesia in gingival recession sites before and in various periods of paste use were determined using diagnostic samples:

### 1) Probing:

a) assessment of tactile sensitivity with cotton wool pads (CWP);
b) linear probe movement over tooth surfaces (LPM).

### 2) Thermometry:

a) irrigation with water jet (WJ):
b) processing with direct air jet (DAJ);
c) processing with side air jet (SAJ).

Tables 6.1 and 6.2 6 provide data on 4-weeks' dynamic pattern of sensitivity built on the basis of diagnostic samples.

TABLE 6.1
Dynamic pattern of sensitivity.

| | Diagnostic tests | | | | | | | | | | | |
|---|---|---|---|---|---|---|---|---|---|---|---|---|
| | In 1 week | | | In 2 weeks | | | In 3 weeks | | | In 4 weeks | | |
| | Compositions | | | | | | | | | | | |
| | A | B | D′ | A | B | D′ | A | B | D′ | A | B | D′ |
| LPM | 8.8 | 8.7 | 8.1 | 16.9 | 16.7 | 12.1 | 22.5 | 25.7 | 16.2 | 31.0 | 32.0 | 20.2 |
| CS | 10.1 | 9.8 | 9.7 | 13.8 | 16.2 | 12.2 | 23.9 | 27.8 | 16.8 | 34.2 | 36.9 | 20.3 |
| WS | 8.4 | 8.2 | 8.1 | 11.4 | 13.4 | 11.9 | 20.2 | 20.1 | 15.0 | 30.3 | 32.8 | 21.2 |
| LAS | 9.3 | 9.1 | 9.1 | 14.0 | 15.2 | 11.8 | 18.2 | 23.6 | 14.1 | 32.4 | 33.2 | 22.3 |
| DAS | 10.1 | 9.7 | 9.6 | 18.6 | 18.7 | 12.1 | 22.2 | 23.9 | 14.2 | 32.9 | 33.2 | 23.3 |

TABLE 6.2
Dynamic pattern of sensitivity.

| | Diagnostic tests | | | | | | | |
|---|---|---|---|---|---|---|---|---|
| | In 1 week | | In 2 weeks | | In 3 weeks | | In 4 weeks | |
| | Compositions | | | | | | | |
| | C | D | C | D | C | D | C | D |
| LPM | 8.9 | 10.0 | 17.1 | 16.1 | 22.9 | 25.8 | 31.4 | 32.3 |
| CS | 10.3 | 12.0 | 13.8 | 16.0 | 24.1 | 28.0 | 34.5 | 36.0 |
| WS | 8.8 | 9.5 | 11.6 | 13.6 | 20.4 | 21.1 | 30.4 | 32.8 |
| LAS | 9.3 | 9.6 | 14.1 | 15.0 | 18.4 | 23.6 | 32.3 | 33.4 |
| DAS | 10.6 | 10.8 | 18.8 | 19.0 | 22.4 | 24.5 | 32.8 | 33.8 |

### 1) Study of Desensitizing Action and Desensitizing Effectiveness.

Teeth sensitivity factor according to L. Yu. Orekhova and S. B. Ulitovskiy (Orekhova-Ulitovskiy TS factor/ [symbol] C3 [symbol] - [symbol] ) allows to trace changes in condition of teeth under the influence of desensitizing agents.

Orekhova-Ulitovskiy TS factor is the total of assessments of all described criteria divided by the number of criteria and multiplied by 100:

$$Orekhova \text{-} Ulitovskiy TS factor = \frac{\sum ( {a}_{1} + … + {a}_{n} )}{5 n} \times 100 \%$$

where X is the total of quantitative assessments of criteria;

a₁ is the score by the first criterion;

aₙ is the score by the n-th criterion;

n is the number of the criteria used;

5 is the quantity of assessed parameters within each criteria.

In the object of the invention the total number of criteria and parameters is stable and equals to 11 and 55, respectively. Thus, the formula will look like:

$$Orekhova \text{-} Ulitovskiy TS factor = \frac{\sum ( {a}_{1} + … + {a}_{n} )}{5 n} \times 100 \%$$

In the denominator the value of total criteria score ranges within 11≦(a₁+ . . . a₁₁)≦55, while bounds the rating are 20≦Orekhova-Ulitovskiy TS factor≦100.

Assessment criteria:

81-100%—highly critical condition;

61-80%—critical condition;

within 41-60%—relatively compensated sensitivity of teeth;

the factor being within 21-40%—relatively compensated sensitivity of teeth, however with underlying compensated mild sensitivity of teeth;

at 20% assessment—a sound tooth (group of teeth) with normal natural sensitivity towards exogenous irritants.

For long-term study of tooth sensitivity in dynamics, especially under influence of drugs or topical use of oral hygiene aids the authors use the formula of calculation of tooth sensitivity efficacy (TS efficacy/ [symbol] C3):

TS efficacy (%)=[(F₁−Fₙ)×100%]/F₁

where F₁ is a numerical value of Orekhova-Ulitovskiy TS factor determined during the first visit:

Fₙ is the numerical value of Orekhova-Ulitovskiy TS factor determined during the n-th visit.

Table 7 summarizes the 4-weeks' history of changes in desensitizing effectiveness according to Orekhova-Ulitovskiy TS factor.

TABLE 7
Changes in desensitizing effectiveness according
to Orekhova-Ulitovskiy TS factor

| Distribution | Examination period | | | |
|---|---|---|---|---|
| by group | Week 1 | Week 2 | Week 3 | Week 4 |
| Composition A | 6.8% | 11.2% | 16.4% | 22.2% |
| Composition B | 6.7% | 11.6% | 17.6% | 21.9% |
| Composition C | 6.8% | 11.4% | 16.5% | 22.1% |
| Composition D | 7.0% | 11.7% | 17.9% | 22.0% |

1) The Study of Whitening Effect and Whitening Effectiveness.

Whitening effect was determined prior and in various times of tooth paste use according to VITAPAN scale. After baseline values were determined according to VITAPAN scale, the authors checked correctness of hygienic care (compliance with toothbrushing technique and conditions), and provided respective training.

Table 8 summarizes the 4-weeks' whitening effect dynamics according to VITAPAN scale.

TABLE 8
Dynamics of whitening effect
according to VITAPAN scale.

| | Examination period | | | | |
|---|---|---|---|---|---|
| Distribution | | In 1 | In 2 | In 3 | In 4 |
| by group | Initial | week | weeks | weeks | weeks |
| Composition | A3.5 | A3.5 | A3 | A3 | A2 |
| A | | | | | |
| Composition | A3.5 | A3.5 | A3 | A3 | A2 |
| B | | | | | |
| Composition | A3.5 | A3.5 | A3 | A2 | A2 |
| C | | | | | |
| Composition | A.3.5 | A3.5 | A3.5 | A3 | A2.5 |
| D | | | | | |
| Composition | A3.5 | A3.5 | A3.5 | A3 | A2.5 |
| D′ | | | | | |

### 1) The Study of Remineralizing Effect and Remineralizing Effectiveness.

Remineralizing effect was studied in TER test according to Okushko. Remineralizing effectiveness was calculated based on TER test.

Table 9 provides data for 4 weeks' changes in remineralizing effectiveness in TER test.

TABLE 9
Changes in remineralizing effectiveness in TER test.

| Distribution | Examination period | | | |
|---|---|---|---|---|
| by group | Week 1 | Week 2 | Week 3 | Week 4 |
| Composition A | 9.3% | 18.0% | 27.1% | 27.2% |
| Composition B | 9.8% | 19.0% | 28.2% | 28.2% |
| Composition C | 9.1% | 18.2% | 27.3% | 27.3% |
| Composition D | 10.0% | 20.0% | 30.0% | 30.0% |
| Composition D′ | 9.8% | 18.8% | 26.7% | 26.9% |

1) The Study of Anti-Inflammatory Effect and Anti-Inflammatory Effectiveness. To Determine the Condition of Periodontal Tissue, PMA Indices Were Used.

Based on findings of PMA indices, the authors determined anti-inflammatory effectiveness which described the nature of changes in inflammatory process in the periodontium.

Anti-inflammatory effectiveness was determined according to the formula:

Anti-inflammatory effectiveness (%)=[100×(PMA₀−PMAₙ)]/PMA₀

where PMA₀ is the numerical value of the index before the start of the study;

PMAₙ is the numerical value of the index in n weeks of the study, at the last visit.

Table 10 provides data for 4 weeks' changes in anti-inflammatory effectiveness of tooth pastes according to PMA index.

TABLE 10
Changes in anti-inflammatory
effectiveness according to PMA index.

| Distribution | Examination period | | | |
|---|---|---|---|---|
| by group | Week 1 | Week 2 | Week | Week 4 |
| Composition A | 6.9% | 13.7% | 22.9% | 40.5% |
| Composition B | 6.4% | 11.6% | 21.1% | 37.9% |
| Composition C | 7.1% | 14.9% | 23.6% | 42.1% |
| Composition D | 5.8% | 10.5% | 21.1% | 38.9% |
| Composition D′ | 5.7% | 10.1% | 18.7% | 36.2% |

### 1) The Study of Hemostatic Effect and Hemostatic Effectiveness.

To determine the condition of periodontal tissue, Muhlemann-Mazor bleeding index was used.

Based on findings of the bleeding index, the authors determined hemostatic effectiveness which showed the nature of changes in the inflammatory process in the periodontium.

Hemostatic effectiveness was determined according to the formula:

Blood-stopping effectiveness (%)=[100×(BI₀−BIₙ)]/BI₀

where BI₀ is the numerical value of the index before the start of the study;

BIₙ is the numerical value of the index in n weeks of the study, at the last visit.

Table 11 provides data of 4-weeks' changes in hemostatic effectiveness using Muhlemaan-Mazor bleeding index.

TABLE 11
Changes in hemostatic effectiveness
using Muhlemann-Mazor bleeding index.

| Distribution | Examination period | | | |
|---|---|---|---|---|
| by group | Week 1 | Week 2 | Week 3 | Week 4 |
| Composition A | 8.1% | 13.1% | 19.8% | 25.1% |
| Composition B | 5.4% | 7.5% | 9.0% | 11.1% |
| Composition C | 8.3% | 13.0% | 20.9% | 26.1% |
| Composition D | 5.5% | 8.5% | 11.0% | 14.5% |
| Composition D′ | 3.2% | 5.4% | 6.8% | 8.1% |

The obtained results allow to conclude that the groups that brushed their teeth-using the claimed composition manifested pronounced cleansing effect, improved sensitivity, desensitizing effectiveness, whitening effectiveness, remineralizing effectiveness, anti-inflammatory effectiveness, and hemostatic effectiveness.

When the reference composition D′ was used, the most parameters were lower, except those that were practically equal.

### Example VIII

### The Comparative Study of Effectiveness of Toothpastes that Contain Glucose Amilase, Alpha-Amilase, Dextranase and Tannase.

Using the technique described in Example I, the toothpaste compositions were made in which tannase was replaced for glucose amilase, alpha-amilase, dextranase, respectively, Composition .formulations coincide with those specified in Table 1 for compositions B and C. Thus the following toothpastes were made:

B and C—with tannase,

B¹ and C¹—with gloucose amylase,

B² and C²—with alpha-amylase,

B³ and C³—with dextranase.

The effectiveness of toothpastes was studied according to the procedure set forth in Example VII.

### 1) The Study of Cleaning Action and Effect.

To determine the cleaning action, the study authors used Green-Vermillion hygienic factor and reagent tablets by PARO.

According to Green-Vermillion hygienic factor, the cleaning effect was determined by the following formula:

Effect (%)=[100×(OHI₀−OHIₙ)]/ OHI₀

where OHI₀ is index value at research start, prior to hygienic procedure;

OHIₙ is index value after n weeks of research, at the last check-up prior to hygienic procedure.

Table 12.1 summarizes 4-week's dynamics data for Green-Vermillion hygienic factor, while Table 12.1, provides data as changes in cleaning efficacy according to Green-Vermillion hygienic factor.

TABLE 12.1
Dynamics of Green-Vermillion hygienic factor.

| | | Examination period | | | | |
|---|---|---|---|---|---|---|
| Pos. | Division into | | 1st | 2nd | 3rd | 4th |
| No. | groups | Start | week | week | week | week |
| 1 | Composition B | 3.72 | 3.23 | 2.84 | 2.43 | 1.27 |
| 2 | Composition C | 3.80 | 3.32 | 2.93 | 2.57 | 2.21 |
| 3 | Composition | 3.77 | 3.33 | 3.01 | 2.55 | 2.45 |
| | B¹ | | | | | |
| 4 | Composition | 3.74 | 3.42 | 3.11 | 2.69 | 2.45 |
| | C¹ | | | | | |
| 5 | Composition | 3.72 | 3.35 | 3.03 | 2.58 | 2.43 |
| | B² | | | | | |
| 6 | Composition | 3.75 | 3.42 | 3.09 | 2.70 | 2.45 |
| | C² | | | | | |
| 7 | Composition | 3.78 | 3.31 | 3.03 | 2.58 | 2.43 |
| | B³ | | | | | |
| 8 | Composition | 3.78 | 3.41 | 3.12 | 2.71 | 2.46 |
| | C³ | | | | | |

TABLE 12.2
Change in the cleaning effect according to the Green-Vermillion factor

| | | Examination period | | | |
|---|---|---|---|---|---|
| Pos. | Division into | 1st | 2nd | 3rd | 4th |
| No. | groups | week | week | week | week |
| 1 | Composition | 13.17% | 23.66% | 34.68% | 38.98% |
| | B | | | | |
| | Composition | 12.63% | 21.89% | 32.37% | 41.84% |
| | C | | | | |
| 3 | Composition | 11.67% | 20.16% | 32.36% | 35.01% |
| | B¹ | | | | |
| 4 | Composition | 8.56% | 16.84% | 28.07% | 34.49% |
| | C¹ | | | | |
| 5 | Composition | 9.95% | 18.55% | 30.65% | 34.68% |
| | B² | | | | |
| 6 | Composition | 8.80% | 17.60% | 28.00% | 34.67% |
| | C² | | | | |
| 7 | Composition | | | | |
| | B³ | 12.43% | 19.84% | 31.75% | 35.71% |
| 8 | Composition | | | | |
| | C³ | 9.79% | 17.46% | 28.31% | 34.92% |

### 1) Study of Sensitivity (Tactile and Heat Samples).

Intensity of hyperesthesia in gingival recession sites before and in various periods of paste use were determined using diagnostic samples:

### 1) Probing:

a) assessment of tactile sensitivity with cotton wool pads (CWP);
b) linear probe movement over tooth surfaces (LPM).

### 2) Thermometry:

a) irrigation with water jet (WJ);
b) processing with direct air jet (DAJ);
c) processing with side air jet (SAJ).

Tables 13 and 14 provide data on 4-weeks' dynamic pattern of sensitivity built on the basis of diagnostic samples.

TABLE 13
Dynamic pattern of sensitivity.

| | In 1 week | | | | In 2 weeks | | | | In 3 weeks | | | | In 4 weeks | | | |
|---|---|---|---|---|---|---|---|---|---|---|---|---|---|---|---|---|
| | Compositions | | | | | | | | | | | | | | | |
| | B | C | B¹ | C¹ | B | C | B¹ | C¹ | B | C | B¹ | C¹ | B | C | B¹ | C¹ |
| LPM | 8.7 | 8.9 | 8.7 | 8.8 | 16.7 | 17.1 | 16.5 | 16.8 | 25.7 | 22.9 | 25.0 | 22.2 | 32.0 | 31.4 | 30.8 | 30.2 |
| CWP | 9.8 | 10.3 | 9.7 | 10.2 | 16.2 | 13.8 | 15.9 | 13.4 | 27.8 | 24.1 | 27.1 | 23.4 | 36.9 | 34.5 | 35.7 | 33.2 |
| WJ | 8.2 | 8.8 | 8.2 | 8.7 | 13.4 | 11.6 | 13.1 | 11.3 | 20.1 | 20.4 | 19.4 | 19.7 | 32.8 | 30.4 | 31.5 | 29.1 |
| SAJ | 9.1 | 9.3 | 9.1 | 9.2 | 15.2 | 14.1 | 14.8 | 13.9 | 23.6 | 18.4 | 22.8 | 17.8 | 33.2 | 32.3 | 32.1 | 30.9 |
| DAJ | 9.7 | 10.6 | 9.6 | 10.5 | 18.7 | 18.8 | 18.5 | 18.5 | 23.9 | 22.4 | 23.0 | 21.8 | 33.2 | 32.8 | 32.0 | 30.9 |

TABLE 14
Dynamic pattern of sensitivity.

| | In 1 week | | | | In 2 weeks | | | | In 3 weeks | | | | In 4 weeks | | | |
|---|---|---|---|---|---|---|---|---|---|---|---|---|---|---|---|---|
| | Compositions | | | | | | | | | | | | | | | |
| | B² | C² | B³ | C³ | B² | C² | B³ | C³ | B² | C² | B³ | C³ | B² | C² | B³ | C³ |
| LPM | 8.7 | 8.9 | 8.6 | 8.7 | 16.6 | 16.7 | 16.4 | 16.7 | 25.1 | 22.8 | 25.2 | 22.3 | 30.5 | 30.5 | 30.6 | 30.6 |
| CWP | 9.7 | 10.1 | 9.5 | 10.0 | 15.8 | 13.2 | 15.7 | 13.2 | 27.3 | 23.0 | 27.4 | 23.2 | 35.5 | 33.5 | 35.3 | 33.6 |
| WJ | 8.1 | 8.9 | 8.1 | 8.6 | 13.2 | 11.5 | 13.2 | 11.1 | 19.4 | 19.7 | 19.5 | 19.9 | 31.6 | 29.3 | 31.2 | 29.3 |
| SAJ | 9.2 | 9.1 | 9.1 | 9.1 | 14.8 | 14.0 | 14.8 | 13.8 | 22.9 | 17.7 | 22.7 | 17.9 | 32.1 | 30.6 | 32.2 | 30.8 |
| DAJ | 9.4 | 10.4 | 9.5 | 10.3 | 18.4 | 18.6 | 18.6 | 18.7 | 23.1 | 21.8 | 23.2 | 21.9 | 32.0 | 30.4 | 32.1 | 30.5 |

### 1) The Study of Whitening Effect and Whitening Effectiveness.

Whitening effect was determined prior and in various times of tooth paste use according to VITAPAN scale. After baseline values were determined according to VITAPAN scale, the authors checked correctness of hygienic care (compliance with toothbrushing technique and conditions), and provided respective training.

Table 15 summarizes the 4-weeks' whitening effect dynamics according to VITAPAN scale.

TABLE 15
Dynamics of whitening effect according to VITAPAN scale.

| Division into | Start | In 1 | In 2 | In 3 | In 4 |
|---|---|---|---|---|---|
| groups | | week | weeks | weeks | weeks |
| Composition B | A3.5 | A3.5 | A3 | A3 | A2 |
| Composition C | A3.5 | A3.5 | A3 | A2 | A2 |
| Composition B¹ | A3.5 | A3.5 | A3 | A3 | A2.5 |
| Composition C¹ | A3.5 | A3.5 | A3 | A2.5 | A2.5 |
| Composition B² | A3.5 | A3.5 | A3 | A2.5 | A2.5 |
| Composition C² | A3.5 | A3.5 | A3.5 | A2 | A2.5 |
| Composition B³ | A3.5 | A3.5 | A3 | A2.5 | A2.5 |
| Composition C³ | A3.5 | A3.5 | A3.5 | A2 | A2.5 |

The obtained results allow to conclude that the groups that used the claimed composition (groups B and C) for tooth brushing showed a pronounced cleaning effect, improved sensitivity and whitening effectiveness.

When the reference compositions containing other enzymes were used, the parameter values were lower; it allows for the conclusion about non-obviousness of use of a hydroxyapatite exactly in combination with tannase enzyme.

### Example IX

### The Comparative Study of Effectiveness of the Toothpastes that Contain Calcium hydroxyapatite.

It is advisable to use a complex containing a hydroxyapatite with 20-80 am particle size. This hydroxyapatite form lacks abrasive properties and has improved biocompatibility with denial tissues.

Toothpaste compositions were made using the: technique described in Example I. The authors studied the compositions containing a hydroxyapatite fraction with 20-80 nm particle size (HAP1) and a hydroxyapatite fraction with 100-150 nm particle size (HAP2). The composition formulations are equal to those specified in Table 1 for C.

### Study of Sensitivity (Tactile and Heat Samples).

Intensity of hyperesthesia in gingival recession sites before and in various periods of paste use were determined using diagnostic samples:

### 1) Probing:

a) assessment of tactile sensitivity with cotton wool pads (CWP);
b) linear probe movement over tooth surfaces (LPM).

### 2) Thermometry:

a) irrigation with water jet (WJ);
b) processing with direct air jet (DAJ);
c) processing with side air jet (SAJ).

Table 16 provides data on 4-weeks' dynamic pattern of sensitivity built on the basis of diagnostic samples.

TABLE 16
Dynamic pattern of sensitivity.

| | In 1 week | | In 2 weeks | | In 3 weeks | | In 4 weeks | |
|---|---|---|---|---|---|---|---|---|
| Compositions | HAP1 | HAP2 | HAP1 | HAP2 | HAP1 | HAP2 | HAP1 | HAP2 |
| LPM | 8.9 | 8.7 | 17.1 | 14.0 | 22.9 | 19.8 | 31.4 | 26.2 |
| CWP | 10.3 | 10.1 | 13.8 | 10.7 | 24.1 | 21.1 | 34.5 | 29.9 |
| WJ | 8.8 | 8.5 | 11.6 | 10.0 | 20.4 | 17.6 | 30.4 | 25.4 |
| SAJ | 9.3 | 8.9 | 14.1 | 11.1 | 18.4 | 15.2 | 32.3 | 27.4 |
| DAJ | 10.6 | 10.2 | 18.8 | 15.6 | 22.4 | 19.1 | 32.8 | 27.5 |

The obtained results allow to conclude that the groups that used the claimed composition (group HAP1) to brush, teeth showed improved sensitivity.

When the reference composition with larger size of amorphous hydroxyapatite the values were lower; it allows for the conclusion about nonobviousness of the use of a hydroxyapatite with max. 80 nm particle size.

Restorative agents for sensitive teeth based on the claimed complex are effective no matter what caused hypersensitivity; expanded dentine tubules, weak dwarfed enamel or exposure of neck areas. Amorphous calcium hydroxyapatite, already after the first application, penetrates deep into lesions on the tooth surface and demineralization loci, thus making enamel more dense and firm, in contrast to agents that block sensitivity of nerve terminals located in dentine tubules or acting superficially. The combined firming effect of toothpastes on the basis of the declared complex is achieved by means of hemostatic, anti-inflammatory, desensiting, remineralizing and anticariogenic effects. One month of using such a toothpaste improves the condition of periodontium; elimination of inflammation and swelling of gums, concretion of soft tissues of gingival margin, no more bleeding gums, and firming of hard tissues of teeth and plugging of open dentine tubules thus ensuring lowered tooth sensitivity. Formation of calcular deposit noticeably reduces (i.e. inhibiting accumulation and growth of soft calcular deposits).

The use of the toothpastes based on the claimed complex allows to achieve such indices as: cleaning effect—up to 77.7%, desensitizing effect—up to 70.4%, remineralizing effectiveness—up to 64.9%, anti-inflammatory effectiveness—up to 67.8%, hemostatic effectiveness—up to 74.1%, and anticariogenic effect—up to 80.8%.

The present invention has been described using detailed descriptions of embodiments thereof that are provided by way of example and are not intended to limit the scope of the invention. The described embodiments comprise different features, not all of which are required in all embodiments of the invention. Some embodiments of the present invention utilize only some of the features or possible combinations of the features. Variations of embodiments of the present invention that are described and embodiments of the present invention comprising different combinations of features noted in the described embodiments will occur to persons of the art. When used in the following claims, the terms “comprises”, “includes”, “have” and their conjugates mean “including but not limited to”. The scope of the invention is limited only by the following claims.

What is claimed is:

1. Mineral-enzymatic complex for cleaning, whitening and firming of tooth enamel and reduction of inflammation and bleeding of gums, the said complex characterized in that it contains tannase and amorphous calcium hydroxyapatite with 20-80 nm particle size, the said complex containing the following components; 0.2-10 weight parts of tannase per 100 weight parts of amorphous calcium hydroxyapatite.
